(19) United States
(12) Patent Application Publication
(10) Pub. No.: US 2010/0312168 A1
(43) Pub. Date: Dec. 9, 2010

(54) LONG LIFE HIGH CAPACITY ELECTRODE, DEVICE, AND METHOD OF MANUFACTURE

(76) Inventors: Yoshimasa Yoshida, Utsunomiya-shi (JP); Masao Ito, Hiratsuka-shi (JP)

Correspondence Address: SEED INTELLECTUAL PROPERTY LAW GROUP PLLC, 701 FIFTH AVE, SUITE 5400, SEATTLE, WA, 98104, US

(21) Appl. No.: 12/796,057

(22) Filed: Jun. 8, 2010

Related U.S. Application Data
(60) Provisional application No. 61/185,544, filed on Jun. 9, 2009.

(51) Int. Cl. A61N 1/30; B23P 19/04

(52) U.S. Cl. 604/20; 29/428

(57) ABSTRACT

Electrodes, particularly electrochemically active electrodes, may benefit from one or more pretreatment cycles in which the electrode is substantially oxidized, reduced or otherwise exhausted prior to use in an end use application, for example active agent delivery via iontophoresis. For instance, electrode lifetime may be advantageously increased, even when used to delivery relatively high currents or used at high current densities. Such may be necessary to delivery therapeutically effect dosage regimes, for instance of oxycodone. Use of a nonwoven fibrous substrate printed with a sacrificial ink may be advantageous relative to other substrates. Use of certain Ag/AgCl inks may be advantageous over other Ag/AgCl inks.

### CROSS REFERENCE TO RELATED APPLICATIONS

This application claims benefit under 35 U.S.C. 119(e) to U.S. provisional patent application Ser. No. 61/185,544, filed 9 Jun. 2009.

### BACKGROUND

1. Technical Field

The present disclosure relates to electrodes and methods of manufacturing electrodes, as well as devices which employ such electrodes, for example medical devices employed in diagnostic and/or related applications, for instance drug delivery via iontophoresis.

2. Description of the Related Art

Electrodes may be used to apply electrical potentials to a circuit, device, or substance from a power source, for instance chemical batteries, fuel cells, super or ultra capacitors, or other exhaustible or non-exhaustible power sources, or from a power supplier, for instance an alternating current (AC) to direct current (DC) rectifier, a DC to AC alternator, a DC to DC converter or transformer and/or other power conditioner. Electrodes may be used to sense electrical characteristics such as current, voltage, resistance or impedance.

Electrodes are used in a large variety of applications from advance industrial electronics to consumer electronics.

Electrodes are particularly useful in medical applications. Electrodes may, for example, be employed to make electrical coupling with biological tissue, for instance with skin, mucous membrane, teeth, bone, heart, brain, nerves, muscles or other biological tissue. Electrodes may be employed to sense physical characteristics, for instance inductance and/or resistance, of various biological tissues, for example to perform an electrocardiogram or electroencephalogram. Such may be achieved externally or internally of a body. Electrodes may also be employed to apply an electrical current through biological tissue, for example to provide electro-stimulation, electrocauterization or electrocicatrization. Such may likewise be achieved externally or internally of a body. Electrodes may also be used to deliver a substance to a biological tissue, for example delivery of an ionized active agent, for instance a drug or other therapeutic or diagnostic substance. Electrodes may also be used to withdraw a substance such as a analyte from a biological tissue. Such delivery or withdrawal may be achieved intradermally or transdermally, for instance via iontophoresis, electroporation and/or other techniques. There are numerous other examples of the use of electrodes in medical end use applications. Some of the end use applications that occur externally may or may not employ a conductive gel or other substance between the electrode and the biological tissue.

Many end use applications may require an electrode to have a relatively high capacity to delivery an electrical potential or high current density. Many end use applications may employ electrochemically active electrodes, for instance sacrificial electrodes. Such may advantageously prevent hydrolysis or electrolysis of water and the formation of undesired reaction products, for instance hydronium ions or hydrogen gases. Such may also advantageously prevent reaction products from interacting with other elements, for instance preventing silver ions or chloride ions from interacting with an active agent such as a drug in an active agent reservoir. Such may additionally or alternatively bind ions that would otherwise compete with ions that are to be delivered, thereby increasing delivery efficiency. However, many end use applications may require an electrode to have a relatively long life time, which may be difficult to achieve with conventional electrochemically active electrodes.

In particular, certain active agents, for example oxycodone, may require a relatively high current density to achieve a therapeutically effective dose. Those same active agents may require delivery over an extended period of time to achieve or maintain a therapeutically effective dose. However, electrochemically active electrodes capable of delivering relatively high current densities often suffer from relatively short lifetimes. While it may be possible to replace such electrodes as the electrodes become exhausted, replacement is a nuisance for a patient or medical service provider who must perform such replacement. Requiring replacement may also reduce compliance with a prescribed dosage regime.

New electrodes and methods to manufacture or pretreat such electrodes, as well as new devices employing such electrodes may provide the desired relatively long electrode life times even when operated at relatively high current densities.

### BRIEF SUMMARY

Electrodes, particularly electrochemically active electrodes, may benefit from one or more pretreatment cycles in which the electrode is substantially oxidized, reduced or otherwise exhausted prior to use in an end use application, for example active agent delivery via iontophoresis. For instance, electrode lifetime may be advantageously increased, even when used to delivery relatively high currents or used at high current densities. Such may be necessary to delivery therapeutically effect dosage regimes, for instance of oxycodone. Use of a nonwoven fibrous substrate printed with a sacrificial ink may be advantageous relative to other substrates. Use of certain Ag/AgCl inks may be advantageous over other Ag/AgCl inks.

A method of manufacture may be summarized as including: prior to an end use application, introducing a first electrode and a second electrode to an electrolytic medium; prior to the end use application, applying an electrical potential of a first polarity to the first electrode and an electrical potential of a second polarity to the second electrode for a first duration of time until at least one of the first and the second electrodes is substantially oxidized or reduced; and incorporating the first and the second electrodes into a device for the end use application.

The method may further include, prior to the end use application and following the first duration of time, applying an electrical potential of the second polarity to the first electrode and an electrical potential of the first polarity to the second electrode for a second duration of time until at least one of the first and the second electrodes is substantially reduced or oxidized. The method may further include, prior to the end use application and following the second duration of time and preceding the incorporating of the first and second electrodes into the device for the end use application, applying an electrical potential of the first polarity to the first electrode and an electrical potential of the second polarity to the second electrode for a third duration of time until at least one of the first and the second electrodes is substantially oxidized or reduced. Introducing a first electrode and a second electrode to an electrolytic medium may include placing the first and the second electrodes in a liquid electrolytic medium. Introducing a first electrode and a second electrode to an electrolytic medium may include moving a first continuous web of electrode material and a second continuous web of electrode material through the electrolytic medium. The end use application may be delivery of an active agent to a biological subject and wherein incorporating the first and the second electrodes into a device for the end use application may include locating one of the first or the second electrodes in an active electrode assembly of the device positioned to selectively apply an electrical potential to an active agent reservoir of the active electrode assembly and locating the other one of the first or the second electrodes in a counter electrode assembly of the device. The method may further include positioning a piece of nonwoven fibrous cloth proximate the first electrode, and loading the piece of nonwoven fibrous cloth with an ionic active agent to be delivered in response to an electrical potential applied to the ionic active agent via the first electrode. The first and the second electrodes may be each part of a respective substrate of material, and may further include, prior to the end use application and preceding the incorporating of the first and the second electrodes into the device for the end use application, separating the first and the second electrodes from the respective substrates of material. The method may further include, prior to the end use application and preceding the introducing of the first and the second electrodes to the electrolytic medium, providing a substrate of nonwoven fibrous material, and depositing a metal/metal salt on the substrate of nonwoven fibrous material to form the first electrode. The method may further include, prior to the end use application and preceding the introducing of the first and the second electrodes to the electrolytic medium, plating a metal/metal salt on a substrate of absorbent nonwoven fibrous material to form the first electrode. The method may further include, prior to the end use application and preceding the introducing of the first and the second electrodes to the electrolytic medium, printing a silver/silver chloride ink on a substrate of absorbent nonwoven fibrous material to form the first electrode. Incorporating the first and the second electrodes into a device for the end use application may occur after introducing the first and the second electrodes to the electrolytic medium and after applying the electrical potential of the first polarity to the first electrode and the electrical potential of the second polarity to the second electrode for the first duration of time. Applying an electrical potential of a first polarity to the first electrode and an electrical potential of a second polarity to the second electrode for a first duration of time until at least one of the first and the second electrodes is substantially oxidized or reduced may include applying the electrical potential of the first polarity to the first electrode until a capacity for oxidation or reduction of the first electrode is reduced by at least 50 percent relative to an oxidation or reduction capacity the first electrode before the first duration of time.

A device for an end use application may be summarized as including: a first electrode which has had an electrical potential of a first polarity applied for a duration of time until the first electrode has been substantially oxidized or reduced at least one before the end use application; a second electrode which has had an electrical potential of a second polarity applied for a duration of time until the second electrode has been substantially oxidized or reduced at least once before the end use application; and a circuit operable to apply a voltage across the first and the second electrodes during the end use application. The electrode may be oxidized or reduced before the electrode is physically associated with a reservoir containing an active agent or drug. Such may advantageously prevent the wasting of active agents such as drugs during pretreatment of the electrode.

The first electrode may have an electrical potential of the second polarity applied for a duration of time until the first electrode has been substantially reconstituted prior to the end use. The first electrode may have an electrical potential of the second polarity applied for a duration of time until the first electrode has been substantially reduced for a second time prior to the end use. The device may further include an active agent reservoir positioned on a delivery side of the first electrode. The device may further include an ionic active agent loaded in the active agent reservoir and selective transportable from the active agent reservoir in response to an electrical potential applied by the first electrode. The device may further include an absorbent nonwoven fibrous cloth active agent reservoir positioned overlaying a biological subject contacting side of the first electrode. The device may further include: an absorbent nonwoven fibrous cloth electrolyte reservoir positioned overlying a biological subject contacting side of the second electrode; and a backing structure that supports the first electrode, the second electrode, the circuit, the absorbent nonwoven fibrous cloth active agent reservoir and the absorbent nonwoven fibrous cloth electrolyte reservoir. The first electrode may have an area of at least 40 cm² and may be capable of delivering 12 mA for 24 hours when driven by the power source. The first electrode may include a nonwoven fibrous cloth and a metal/metal salt material. The metal/metal salt material may include an Ag/AgCl mixture of at least 50 mg/cm³. The first electrode may include at least one of a metal foil or screen and has a thickness of at least 30 μm.

A device for an end use application of drug delivery may be summarized as including: a first sacrificial electrode; a second electrode; an drug reservoir positioned on a delivery side of the first electrode to be responsive to an electrical potential applied by the first sacrificial electrode to delivery a drug from the drug reservoir to a biological interface; and a circuit operable to apply a voltage from a power source across the first and the second electrodes during the end use application, wherein the first sacrificial electrode has a capacity of delivering 0.3 mA/cm² for a 24 hour duration when driven by the circuit.

The first sacrificial electrode may be sized to delivery 12 mA for 24 hours when driven by the power source. The first sacrificial electrode may have an area of at least 40 cm². The first sacrificial electrode may include Ag/AgCl. The first sacrificial electrode may include a piece of nonwoven fibrous cloth bearing an Ag/AgCl mixture. The first sacrificial electrode may include a piece of polymer substrate bearing an Ag/AgCl mixture. The first sacrificial electrode may include a piece of nonwoven fibrous cloth bearing an Ag/AgCl mixture of at least 50 mg/cm³. The first sacrificial electrode may include a piece of foil having a thickness of at least 20 μm and the first sacrificial electrode may have had an electrical potential of a first polarity applied for a first duration of time and an electrical potential of a second polarity applied for a second duration of time until the first electrode has been substantially oxidized at least once and reduced at least once before the end use application. The first sacrificial electrode may include a piece of foil having a thickness of at least 30 μm and the first sacrificial electrode may have had an electrical potential of a first polarity applied for a first duration of time and an electrical potential of a second polarity applied for a second duration of time until the first electrode has been substantially oxidized at least once and reduced at least once before the end use application. The first sacrificial electrode may include a piece of foil having a thickness of at least 50 μm. The Ag/AgCl mixture may have had an electrical potential of a first polarity applied for a first duration of time until the first electrode has been substantially oxidized at least one and may have had an electrical potential of a second polarity, opposite the first polarity, applied for a second duration of time until reduced at least once before the end use application. The device may further include a therapeutically effective quantity of oxycodone stored in the drug reservoir.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1A is a schematic diagram showing a pair of electrodes in an electrolytic medium and a device for pretreating the electrodes prior to an end use application, according to one illustrated embodiment.

FIG. 1B is a side elevational view of a nonwoven fibrous material type electrode, according to one illustrated embodiment.

FIG. 1C is an isometric view of a foil type electrode, according to one illustrated embodiment.

FIG. 1D is an isometric view of a screen type electrode, according to one illustrated embodiment.

FIG. 1E is an isometric view of a plate type electrode, according to one illustrated embodiment.

FIG. 2 shows an exploded isometric view of a medical device for an end use application, the device employing electrodes that have been pretreated prior to an end use application, according to one illustrated embodiment.

FIG. 3 is an isometric view showing successive manufacturing operations to produce pretreated electrodes using a continuous web manufacturing processes, according to one illustrated embodiment.

FIG. 4 is a schematic view showing a manufacturing operation to produce a medical device employing pretreated electrodes using a continuous web manufacturing process, according to one illustrated embodiment.

FIG. 5 is a flow diagram of a method of pretreating electrodes, according to one illustrated embodiment.

FIG. 6 is a flow diagram of a method of pretreating electrodes which may be employed in addition to the method illustrated in FIG. 5.

FIG. 7 is a flow diagram of a method of pretreating electrodes which may be used in addition to the method of FIG. 6.

FIG. 8 is a flow diagram of a portion of a method of pretreating electrodes which may be used as part of the method of FIG. 5.

FIG. 9 is a flow diagram showing a method of forming electrodes, according to one illustrated embodiment.

FIG. 10 is a flow diagram showing a method of incorporating electrodes in an end user application device, according to one illustrated embodiment.

FIG. 11 is a flow diagram showing a method of forming a medically related device for delivering an active agent to a biological interface, according to one illustrated embodiment.

FIG. 12 is a flow diagram showing a method of forming an electrode, prior to pretreatment of the electrode, according to one illustrated embodiment.

FIG. 13 is a flow diagram showing a method of forming an electrode, prior to pretreatment of the electrode, according to another illustrated embodiment.

FIG. 14 is a flow diagram showing a method of forming an electrode, prior to pretreatment of the electrode, according to yet another illustrated embodiment.

FIG. 15 is a flow diagram showing a method of forming an electrode, prior to pretreatment of the electrode, according to still another illustrated embodiment.

FIG. 16 is a flow diagram showing a method of forming an electrode, prior to pretreatment of the electrode, according to a further illustrated embodiment.

FIG. 17 is a flow diagram showing a method of forming a medical device for a medical end use application, the medical device employing electrodes to transfer one or more active agents to a biological interface as the end use application, according to one illustrated embodiment.

FIGS. 18A-18C are scanning electron micrographs showing a first type of nonwoven fibrous or cloth type electrode before and after subsequently applications of electrical potentials thereto.

FIGS. 19A-19C are scanning electron micrographs showing a second type of nonwoven fibrous or cloth type electrode before and after subsequently applications of electrical potentials thereto.

FIGS. 20A and 20B are scanning electron micrographs showing a first type of nonwoven fibrous or cloth electrode employing DuPont Ink, that has been subjected to pretreatment.

FIGS. 21A and 21B are scanning electron micrographs showing a second type of nonwoven fibrous or cloth electrode, that has been subjected to pretreatment.

FIGS. 22A and 22B are scanning electron micrographs showing ink coating nonwoven fibers of a nonwoven substrate type electrode.

FIGS. 23A and 23B are scanning electron micrographs showing an electrode before and after pretreatment, respectively.

FIGS. 24A and 24B are scanning electron micrographs showing an electrode before and after pretreatment, respectively.

FIG. 25 is a chart showing the results testing of nonwoven cloth and PETE substrate type electrodes.

FIG. 26 are charts showing the results of testing pretreating of electrodes at a number of different current densities.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with electrodes, medical devices for example iontophoresis devices, and/or continuous web manufacturing machinery have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

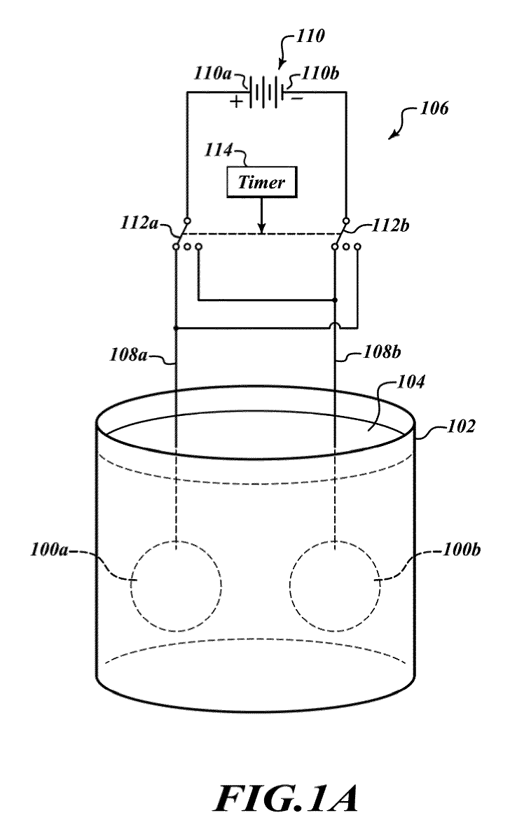

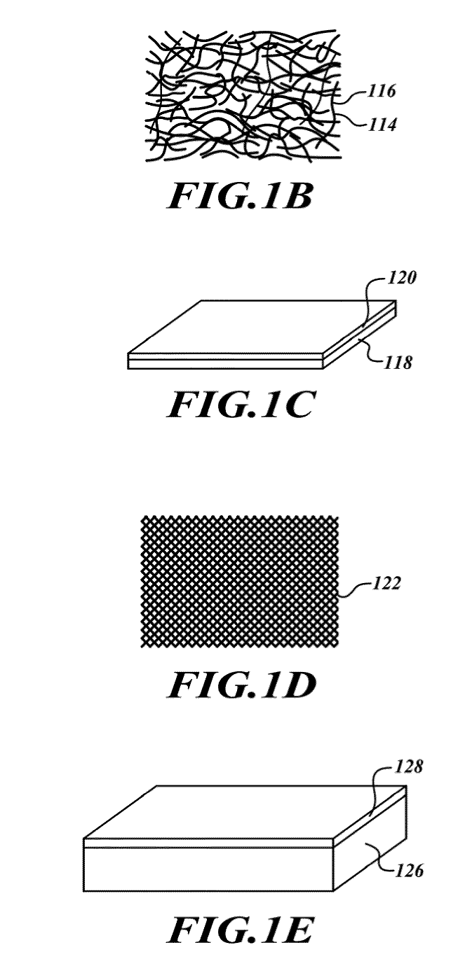

FIG. 1A shows a first electrode 100a and a second electrode 100b (collectively 100) being pretreated prior to an end use application, according to one illustrated embodiment.

The electrodes 100 may advantageously be electrochemically active electrodes, for example sacrificial electrodes. As used herein and in the claims the term “sacrificial” means that at least a portion of an electrode (e.g., anode or cathode) is electrochemically oxidizable or reducible via a reduction/oxidation (i.e., redox) reaction. As used herein and in the claims, “electrochemically active” means sacrificial materials, as well as materials that participate in the redox reaction but which are not themselves oxidized or reduced during the redox reaction. As previously noted, electrochemically active electrodes may advantageously reduce the formation of undesired reaction products, prevent reaction products from interfering with other elements such as active agents to be delivered, and may increase the transport efficiency of active agent delivery by reducing competing species of ions.

As illustrated in FIG. 1A, the electrodes 100 are located in a vessel 102 containing an electrolyte medium 104. The electrolyte medium may be a fluid, for example a liquid, vapor or gas, which provides a medium to support redox reactions between the electrodes 100a, 100b. Any of a large variety of known electrolytic mediums may be suitable.

As further illustrated in FIG. 1A, a pretreatment circuit 106 may be employed to selectively apply electrical potentials to the electrodes 100 to pretreat the electrodes 100. As used herein and in the claims, the terms “pretreatment” “pretreat” and “pretreating” mean an application of an electrical potential to an electrode to cause a reversible redox reaction that substantially reduces, oxidizes, or otherwise exhausts the electrode 100 prior to or before use of the electrode 100 in an end use application. Thus, “pre” is used to denote a treatment that occurs before an end use application. As used herein and in the claims, “end use application” means the intended use or application of the device, which use is separate and distinct from the manufacture of electrode 100 or device in which the electrode may be incorporated. For example, where the electrodes 100 will be incorporated in a medical device, the end use may be a use on a patient or subject. For instance, where the electrodes are incorporated in an iontophoresis device, the end use application may be delivery of an active agent (e.g., therapeutic or diagnostic substance) to a biological tissue of a biological subject (e.g., human or other animal) or withdrawal of a substance therefrom. Alternatively, the end use may be the sensing or measurement of a physical characteristic of the biological tissue of the patient or subject. Alternatively, the end use may be the application of an electrical current to the biological tissue of a patient or subject. Pretreatment constitutes the application of an electrical potential to the electrodes 100, prior to the end use of the electrodes to deliver active agent or electrical current to the biological tissue or to sense or measure physical characteristics thereof. Pretreatment of the electrodes may advantageously occur before the electrodes are physically associated with an active agent or “drug” containing reservoir to prevent wastage of active agent or drug during the pretreatment. As used herein and in the claims, the term “substantially,” when used to modify the terms “oxidize,” “reduced,” or “exhausted” means a change of 50% or more in an ability of the electrode to deliver an electrical potential, unless otherwise specified.

As illustrated, the pretreatment circuit 106 includes a first lead 108a which is electrically and optionally physically coupled to the first electrode 100a. The pretreatment circuit 106 also includes a second lead 108b which is electrically and optionally physically coupled to the second electrode 100b. The first and second leads, collectively 108, may be electrically and physically coupled to the electrodes 100 via respective clips, for instance alligator clips, or other selectively releasable structures.

The pretreatment circuit 106 includes one or more power sources 110 having a first pole 110a of a first polarity and a second pole 110b of a second polarity, opposite the first polarity. The power source 110 may take a variety of forms, for example an exhaustible power storage device or an inexhaustible power production device. For instance, the power source 100 may take the form of an array of chemical cells (e.g., battery), array of super- or ultra-capacitors, array of fuel cells, etc. Also for instance, the power source 100 may take the form of a power supply, for example a direct current (DC) power supply which receives power from a source such as a grid or electrical outlet receptacle. The power supply may include a rectifier to rectify alternating current to DC power, a transformer or DC/DC converter (e.g., step down converter) to adjust a voltage of the electrical power, as well as one or more power conditioner circuits.

The pretreatment circuit 106 also includes one or more switches 112a, 112b (collectively 112) selectively operable to couple the electrodes 100 to the poles 100a, 100b of the power source 110. For example, a double throw switch may be employed. In a first position, the switch 112 may electrically couple the first electrode to a positive pole 110a of the power source 110 and electrically couple the second electrode to the negative pole 110b of the power source 110 during a first duration or period of time. In a second position, the switch 112 may electrically couple the first electrode to the negative pole 110b of the power source 110 and electrically couple the second electrode 100b to the positive pole 110a of the power source during a second duration or period of time. The first duration may be sufficiently long to substantially oxidize, reduce and/or exhaust at least one of the electrodes 100. The second duration may be sufficiently long to substantially oxide, reduce, or exhaust at least one of the electrodes 100.

Optionally, in a third position, the switch 112 may electrically uncouple both the first and the second electrodes 110a, 110b from both the positive and negative poles 110a, 110b of the power source 110 and at the same time. Such allow the electrodes 100 to be uncoupled from pretreatment circuit 106, for example at the start and end of pretreatment, or between successive pretreatment applications of electrical potentials.

In some embodiments, the switch 112 may be used to apply the first electrode 100a to the positive pole 110a of the power source and the second electrode 100b to the negative pole 110b of the power source during a third duration or period of time.

While in some embodiments the switch 112 may be manually operated, in other embodiments a timer 114 or other control circuit may be employed to control the switching or position of switch 112. Such may allow the pretreatment protocol to be automated, which may reduce costs and produce more consistent results. The timer 114 may be implement in a programmed general purpose computer, such as a PC computer, or may be implemented as a special purpose pretreatment controller employing a processor, microcontroller, gate array, application specific integrated circuit or other control circuit, with or with memory or storage medium.

Electrodes 100 may take a variety of forms, shapes, sizes, and/or may comprise a variety of materials.

For example as illustrated in FIG. 1B electrodes 100 may take the form of a fibrous material that comprises a plurality of fibers 116 and carries a metal/metal salt material 118. Such fibrous material may take the form of a nonwoven fibrous cloth material. The metal/metal salt material 118 may be deposited on the nonwoven fibrous cloth material in a variety of ways. For example, the metal/metal salt may be deposited as a coating of a metal/metal salt ink. The metal/metal salt may advantageously be printed on the nonwoven fibrous material. The metal/metal salt may form a layer on a surface of the fibrous material or may individually coat individual fibers, extending into an interior of the nonwoven fibrous material. For example, the metal/metal salt may be advantageously screen printed on the nonwoven fibrous material, for example at least partially coating individual fibers. Without being bound by theory, such may achieve a particularly high amount of exposed metal/metal salt surface area, the metal/metal salt coating individual fibers making up the nonwoven fibrous material. Such may provide enhanced performance relative to other electrode structures.

Alternatively as illustrated in FIG. 1C, electrodes 100 may take the form of a foil 118. The foil 118 may be a metal. The metal of the foil 118 may be a sacrificial material or may simply provide a support for a sacrificial material. Hence, the foil 118 may comprise the metal salt material or may carry a metal salt or a metal/metal salt material 120 deposited thereon. A foil 118 may achieve relatively high performance, and may be relatively inexpensive or simpler to manufacture.

Alternatively as illustrated in FIG. 1D, electrodes 100 may take the form of a screen 122. The screen 122 may formed metal wires. The metal of the screen 122 may be a sacrificial material or may simply provide a support for a sacrificial material. Alternatively, the screen may be formed of another material, for example a polymer material such as polyethylene terephthalic ester (i.e., PETE) which carries a metal/metal salt material. Hence, the screen comprise a metal salt or may carry metal salt or a metal/metal salt material deposited on individual fibers or wires thereof. A screen has a relatively large exposed area compared to a foil, depending on the mesh size of the screen. Thus, a screen 112 type electrode may be able to achieve relatively high performance relative to a foil type electrode, and may be relatively inexpensive or simpler to manufacture. However, a screen 122 will likely provide a smaller exposed surface area than a nonwoven fibrous material type electrode.

Alternatively as illustrated in FIG. 1E, electrodes 100 may take the form of a metal plate 126. The metal of the metal plate 126 may be a sacrificial material or may simply provide a support for a sacrificial material. Hence, the metal plate may carry a metal salt or metal/metal salt material 128 deposited thereon.

Electrodes 100 may take the form of a polymer substrate, for example a polyethylene terephthalic ester (PETE) substrate, coated with a metal/metal salt material. Such may not be as advantageous as previously described embodiments of the electrodes 100.

As previously stated, electrodes 100 that are electrochemically active or sacrificial may comprises a metal/metal salt. Suitable metals may include silver, copper, molybdenum, which form insoluble halide salts (AgCl, Agl, AgBr, CuCl, Cul, CuBr, MoCl₃, MoL₂). Such metals may be particularly suitable for an anode for use in the delivery of cationic active agents. An anode of silver may be particularly advantageous since silver chloride is highly insoluble and many cationic drugs are commercially available in hydrochloride forms. Where Ag/AgCl is employed, the redox reaction transforms the silver of the anode into silver chloride by oxidation, and transforms the silver chloride of the cathode into silver by reduction. In some embodiments, the electrodes 100 may include other non-metal or non-metal salt materials, for example carbon or carbon fiber.

Some examples of suitable dimensions are set out in the various examples, below.

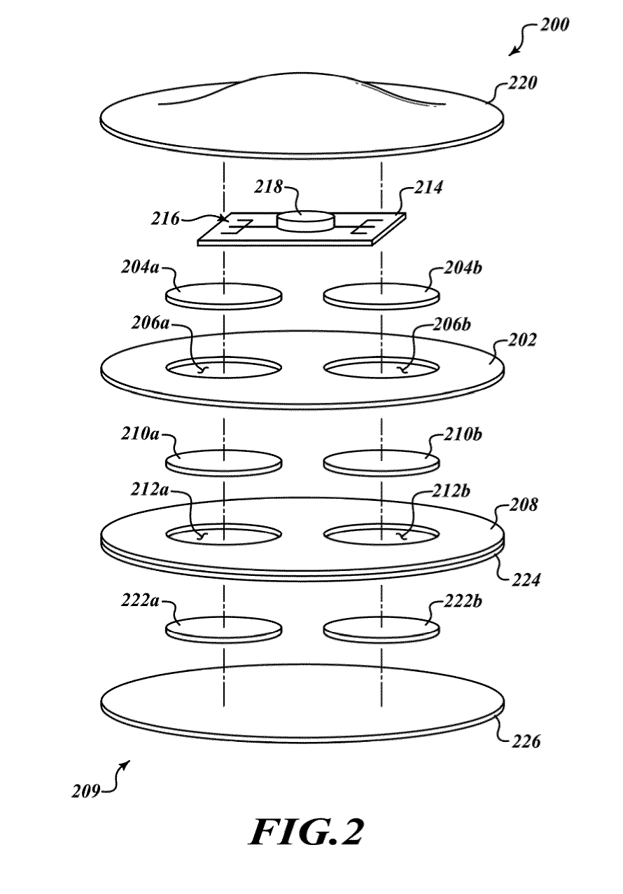

FIG. 2 shows a medical device employing electrodes, in the form of an iontophoresis device 200, according to one illustrated embodiment.

The iontophoretic device 200 may include a first substrate 202 such as a backing tape. A pair of electrodes 204a, 204b (collectively 204) may be mounted aligned with or within apertures 206a, 206b formed in the first substrate 202.

The iontophoretic device 200 may include a second substrate 208 such as a backing tape. The second substrate 208 may be disposed towards a biological tissue contacting side 209 of the iontophoretic device 200 relative to the first substrate 202. Reservoirs 210a, 210b (collectively 210) may be mounted aligned with or within apertures 212a, 212b of the second substrate 208, respectively, and hence aligned with electrodes 204a, 204b, respectively. One of the reservoirs 210a may function as an active agent reservoir, storing an active agent such as a drug or other therapeutic or diagnostic material to be delivered as part of the end use application. The other reservoir 210b may function as an electrolyte reservoir storing an electrolyte to facilitate the end use application (e.g., delivery of active agent or withdrawal of specimen or analyte from the bodily tissue).

The iontophoretic device 200 may include a circuit board 214, for instance a flexible circuit board (e.g., FR4) that carries a control or delivery circuit 216 and optionally a power source 218, which may include printed circuit traces thereon or therein. The control or delivery circuit 216 is configured to electrically couple the power source 218 to respective ones of the electrodes 204. The control or delivery circuit 216 may control the current and/or voltage applied to the respective electrodes 204, and hence control a delivery profile over time of the active agent. While the power source 218 is illustrated as being carried directly on the circuit substrate 214, in other embodiments the power source 218 may be external from, and selectively attachable to, the remainder of the iontophoretic device 200. For example, a power source 218 such as a chemical battery cell, may be electrically and physically coupled to the remainder of the iontophoretic device 200 using a snap, clip, or one or more magnets, for instance as described in U.S. Patent Application Publication No. 2008-0154178.

Control or agent delivery regime or profile may be specified and/or implemented by a wide range of hardware, software, firmware, or virtually any combination thereof. In one embodiment, control may be implemented via Application Specific Integrated Circuits (ASICs). However, those skilled in the art will recognize that the embodiments disclosed herein, in whole or in part, can be equivalently implemented in standard integrated circuits, as one or more computer programs executed by one or more computers (e.g., as one or more programs running on one or more computer systems), as one or more programs executed by on one or more controllers (e.g., microcontrollers) as one or more programs executed by one or more processors (e.g., microprocessors), as firmware, or as virtually any combination thereof, and that designing the circuitry and/or writing the code for the software and or firmware would be well within the skill of one of ordinary skill in the art in light of the teachings of this disclosure.

When logic is implemented as software and stored in memory, logic or information can be stored on any computer-readable medium for use by or in connection with any processor-related system or method. In the context of this disclosure, a memory is a computer-readable medium that is an electronic, magnetic, optical, or other physical device or means that contains or stores a computer and/or processor program. Logic and/or the information can be embodied in any computer-readable medium for use by or in connection with an instruction execution system, apparatus, or device, such as a computer-based system, processor-containing system, or other system that can fetch the instructions from the instruction execution system, apparatus, or device and execute the instructions associated with logic and/or information.

In the context of this specification, a “computer-readable medium” can be any physical element that can store a program associated with logic and/or information for use by or in connection with the instruction execution system, apparatus, and/or device. The computer-readable medium can be, for example, but is not limited to, an electronic, magnetic, optical, electromagnetic, infrared, or semiconductor system, apparatus or device. More specific examples (a non-exhaustive list) of the computer readable medium would include the following: a portable computer diskette (magnetic, compact flash card, secure digital, or the like), a random access memory (RAM), a read-only memory (ROM), an erasable programmable read-only memory (EPROM, EEPROM, or Flash memory), a portable compact disc read-only memory (CDROM), digital tape.

A cover or other protective layer 220 may overlie the circuit substrate 214. The cover 220 may take the form of a polymer sheet, and may be hermetically sealed to the first substrate 202 to provide environmental protection to the circuit substrate 214, control or delivery circuit 216, electrodes 204 and/or reservoirs 210.

The iontophoretic device 200 may include scrims 222a, 222b (collectively 222) which may be received over, in, or partially in, respective apertures 212a, 212b of the second substrate 208. Scrims may take a variety of forms, including a nonwoven fibrous material such as nonwoven cloth material.

The iontophoretic device 200 may include a pressure sensitive adhesive 224 which allows the iontophoretic device 200 to be securely fastened to a biological interface. The pressure sensitive adhesive may, for example, be carried by the second substrate 208. The iontophoretic device 200 may include a selectively releasable release liner 226 which may cover the scrims 222 and pressure sensitive adhesive 224. Removal of the release liner exposes the pressure sensitive adhesive 224, allowing application of the iontophoretic device 200 directly to the biological tissue. Alternatively, a conductive medium such as a conductive gel medium may be employed between the iontophoretic device 200 and the biological tissue.

FIG. 3 shows a manufacturing environment 300 to produce pretreated electrodes, according to one illustrated embodiment.

Electrode substrate material 302 may be supplied as a continuous web via a supply roll 304, which may be advanced via a take-up roll 306 driven by one or more motors 308.

The electrode substrate material 302 may take a variety of forms. As described above and in detail further below, the electrode substrate material 302 may advantageously take the form of an absorbent, nonwoven fibrous material such as a nonwoven cloth material. The use of such nonwoven fibrous material may provide results that are surprisingly better than that realized of other materials. Alternatively, the electrode substrate material 302 may take the form of a polymer such as a polyethylene terephthalate (PET). Alternatively, the electrode substrate material 302 may take the form of a foil such as a metal foil, or a screen such as a metal screen or a plate such as a metal plate.

An application device, for example a screen printer 310, may deposit, print, spray or otherwise apply a metal/metal salt material on the electrode substrate material 302. The metal/metal salt material may come from a reservoir 312 supplied via a valve 314 and conduit 316. As previously identified, the metal/metal salt may take a variety of forms. For example, the metal/metal salt may advantageously take the form of silver/silver chloride (Ag/AgCl). A particularly suitable silver/silver chloride material may be commercially available from DuPont under the designation Ag/AgCl Ink No. L-8144. The result is a continuous web of electrode 320 taken up by take-up roll 306. Multiple rolls of the continuous web of electrode 320 may be manufactured in advance or pretreatment of the continuous web electrode 320.

Continuous webs 320a, 320b (collectively 320) of electrodes may be conveyed through a reservoir 322 containing an electrolyte medium 324 for pretreatment. The continuous webs 320 may be supplied from supply rolls 326a, 326b (collectively 326) and drawn via take-up rolls 328a, 328b (collectively 328) driven by one or more motors 330a, 330b (collectively 330). Notably, the electrodes have not yet been physically associated with active agent or drug reservoirs containing active agents or drugs. Hence, pretreatment will not waste drugs or other active agents. Alternatively, in some embodiments, pretreatment may occur after physical associating the electrodes with reservoirs, but those reservoirs are preferably empty or devoid of active agents such as drugs to avoid the wastage of such materials.

One or more pretreatment circuits 332 may apply electrical potentials to the continuous web of electrode 320 while in the electrolyte medium 324. For example, the pretreatment circuit 332 may apply electrical potentials via one or more pads 334a, 334b (collectively 334). The pads 334 may be flat with a relatively smooth finish or may have a textured surface, for example numerous electrically conductive fibers. The pretreatment circuit 332 may apply electrical potentials to substantially reduce, oxidize, or exhaust the electrodes 320 at least once. In some embodiments, the pretreatment circuit 332 may subsequently reduce or oxidize, or substantially exhaust the electrodes a second, third, or more times, each time reversing a polarity of the electrical potential applied to the respective continuous webs of electrode 320a, 320b. Thus for example the continuous webs of electrode 320 may be run in one direction a first time and then in the opposite direction a second time, with a polarity of the electrical potential applied being switched back and forth with each direction change. Alternatively, the take-up rolls 328 may be removed and used as supply rolls 326 in a second pass of the continuous webs electrode 320 through the electrolyte medium 324. The continuous web of electrode 320 may be manufactured by a separate entity from the entity that performs the pretreatment, or may be manufactured by the same entity as the entity that performs the pretreatment.

The resulting pretreated electrode material 340a, 340b (collectively 340) taken up on take-up rolls 328 may be processed into discrete electrodes 342. For example, the pretreated electrode material 340 may be die-cut, laser cut, or otherwise cut into individual electrodes 342 (only two called out in FIG. 3), for example via a die-cutting apparatus 344. The die-cutting apparatus 344 may include a die 346, a platen 348 to support the pretreated electrode material 340, and a pneumatic actuator 350 controlled by a controller 352 which supplies pressure from a pneumatic source 354 via one or more valves 356. The formation of individual electrodes 342 from the continuous webs of pretreated electrode 340 may be performed by a separate entity from the entity that manufactures the continuous webs of electrode 320 and/or that performs the pretreatment, or may be performed by the same entity as the entity that either manufactures the continuous webs of electrode 320 or that performs the pretreatment.

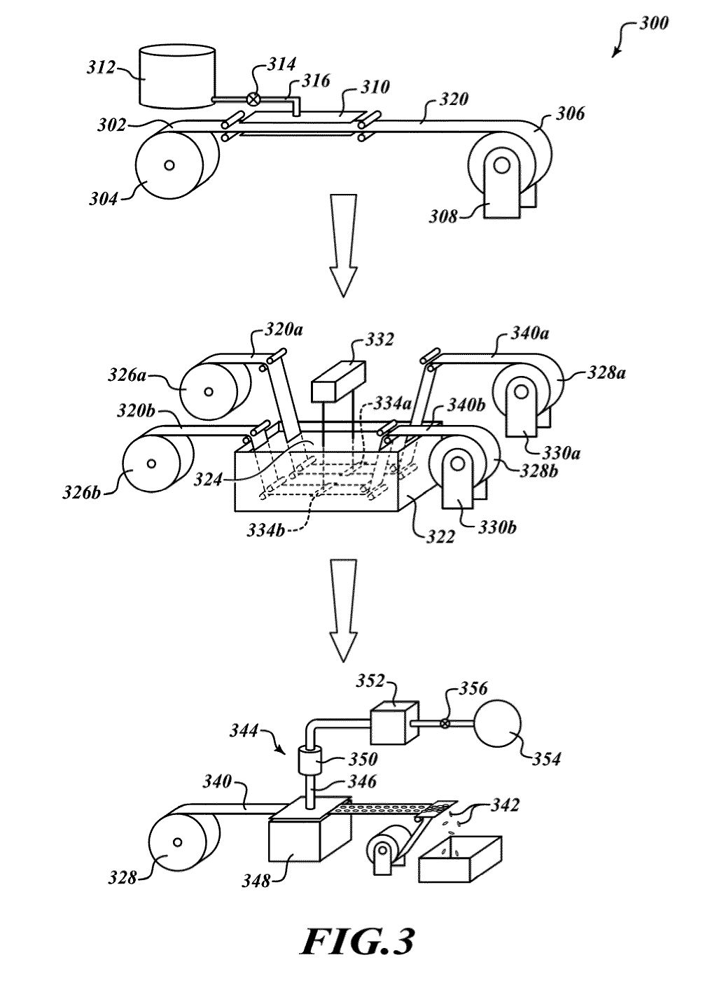

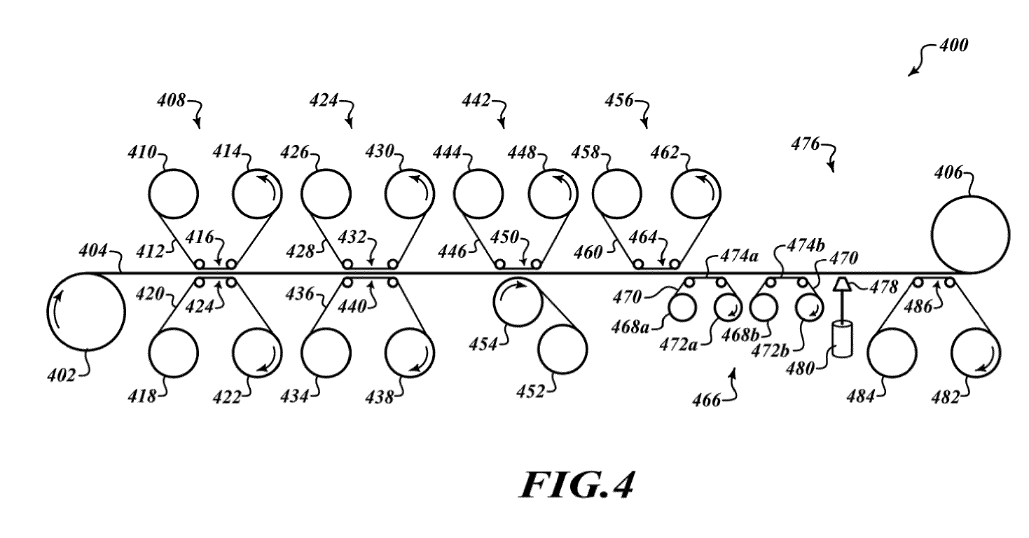

FIG. 4 shows an environment 400 for manufacturing medical devices employing pretreated electrodes, according to one illustrated embodiment.

The environment 400 may advantageously employ continuous web manufacturing processes and structures. For example, a supply roll 402 may supply a first substrate 404, for instance backup tape, to a take-up roll 406 which may be driven by a motor (not shown in FIG. 4). The environment may include various rollers, conveyors and other transport mechanisms, which are omitted from FIG. 4 for sake of clarity of illustration. One or more manufacturing operations may take place using the substrate 404 as a base. Some of these manufacturing operations are described below, although additional or different operations may be included, and some of the described operations may be omitted. The various operations are discussed with respect to various stations. Some embodiments may not employ specific stations, or may have stations that overlap. Further, many of the operations and/or stations may occur in a different order than illustrated in FIG. 4.

At a first station 408, a first electrode supply roll 410 supplies a continuous web of pretreated first electrodes (e.g., cathodes) 412 via a take-up roll 414. An apparatus such as a separator and/or application 416 may separate individual electrodes from the continuous web 412 and apply the electrodes to the first substrate 404. The applicator 416 may take the form of a press which may employ electrically conductive adhesive, pressure and/or heat to apply the electrodes to the first substrate 404 at desired locations.

At the first station 408, a first reservoir supply roll 418 may supply a continuous web of reservoirs 420 via a take-up roll 422. An apparatus such as a separator and applicator 424 may separate and/or apply reservoirs to the first substrate 404. The applicator 424 may take the form of a press which may employ electrically conductive adhesive, pressure and/or heat to apply the first reservoirs to the first substrate 404 at desired locations, for example aligned or in registration with respective ones of the first electrodes. The first reservoirs may, for example, be employed as active agent reservoirs.

At a second station 424, a second electrode supply roll 426 supplies a continuous web of pretreated second electrodes (e.g., anode) 428 via a take-up roll 430. An apparatus such as a separator and/or applicator 432 may separate individual electrodes from the continuous web 428 and/or apply the electrodes to the first substrate 404. The applicator 432 may take the form of a press which may employ electrically conductive adhesive, pressure and/or heat to apply the electrodes to the first substrate 404 at desired locations relative to the first electrodes.

At the second station 424, a second reservoir supply roll 434 supplies a continuous web of reservoirs 436 via a take-up roll 438. An apparatus such as a separator and/or applicator 440 may separate and/or apply the reservoirs to the first substrate 404 aligned with the second electrode. The applicator 440 may take the form of a press which may employ electrically conductive adhesive, pressure and/or heat to apply the second reservoirs to the first substrate 404 at desired locations, for example aligned or in registration with respective ones of the second electrodes. The second reservoir may, for example, be employed as electrolyte reservoirs.

At a third station 442, a circuit board supply roll 444 may supply a continuous web of flexible circuit board and/or power source (e.g., battery) 446 via a take-up roll 448. An apparatus such as a separator and/or applicator 450 may separate discrete flexible circuit boards from the continuous web 446 and apply such to the first substrate 404. The applicator 450 may take the form of a press which may employ electrically conductive adhesive, pressure and/or heat to apply the circuit boards to the first substrate 404 at desired locations, for example aligned or in registration with respective ones of the first and second electrodes.

At the third station 442, a second substrate supply roll 452 may supply a second substrate, for example, a second backing tape, via a take-up roll 454. The second substrate may be applied to the first substrate. In some embodiments, the second substrate may be applied before the first and/or second reservoirs.

At a fourth station 456, a cover supply roll 458 may supply a continuous web of a cover material 460 via a take-up roll 462. An apparatus such as a separator and/or applicator 464 may separate, cut, and apply the cover to the substrate 404. The applicator 464 may take the form of a press which may employ electrically conductive adhesive, pressure and/or heat to apply the covers to the first substrate 404 at desired locations, for example overlying the circuit boards. In particular, the cover may be applied to provide environmental protection to the flexible circuit substrate.

At a fifth station 466, first and second scrim supply rolls 468a, 468b may supply scrim material 740 via take-up rolls 472a, 472b. An apparatus such as a separators and/or applicators 474a, 474b (collectively 474), may separate discrete scrim elements and apply them to the substrates overlying the respective reservoirs. The applicator 474 may take the form of a press which may employ adhesive, pressure and/or heat to apply the scrims over the reservoirs.

At a sixth station 476, one or more nozzles or jets 478 may apply a pressure sensitive adhesive to a biological tissue facing side of the medical device. The pressure sensitive adhesive may be supplied from a reservoir of pressure sensitive adhesive 480. A liner supply roll 482 may supply a continuous web of release liner via a take-up roll 484. An apparatus such as applicator 486 may apply the release liner over the pressure sensitive adhesive. The applicator may employ a press or heat.

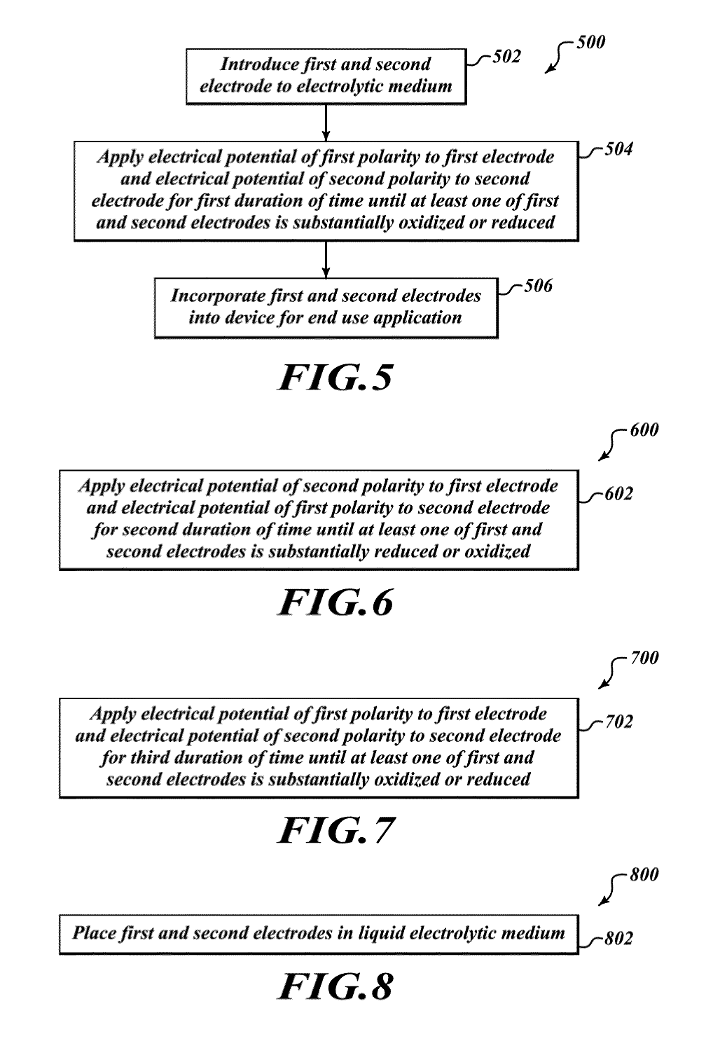

FIG. 5 shows a method of producing pretreated electrodes, according to one illustrated embodiment.

At 502, a first and a second electrode are introduced into electrolytic medium. As previously noted, the electrodes may take a variety of forms, for example, an absorbent nonwoven cloth material, a polymer material such as a polyethylene, a metal material such as a foil or screen. The electrode may comprise a sacrificial material, for example, a metal/metal salt material. Suitable metal/metal salt materials may take a variety of forms including silver/silver chloride (Ag/AgCl).

Prior to an end use application, at 504 an electrical potential of a first polarity is applied to the first electrode and an electrical potential of a second polarity, opposite the first polarity, is applied to the second electrode. The electrical potentials are applied for a first duration or period of time, until at least one of the first and second electrodes is substantially oxidized, reduced, or exhausted.

At 506, the first and second electrodes are incorporated into an end use device intended for an end use application. For example, the electrodes may be incorporated into a medical device for a medical end use application. For instance, the electrodes may be incorporated into an iontophoresis device for providing iontophoretic delivery of an active agent such as a drug.

Notably, all of the above may be accomplished before physically associating the electrodes with any active agent or drug reservoirs, or before loading drugs or other active agents in such reservoirs.

FIG. 6 shows a method 600 of pretreating an electrode, according to another illustrated embodiment. Method 600 may be employed in addition to the method 500 (FIG. 5).

Prior to an end use application and following 504 (FIG. 5), at 602 an electrical potential of the second polarity is applied to the first electrode and an electrical potential of the first polarity is applied to the second electrode. The electrical potentials may be applied for a second duration or period of time, until at least one of the first or second electrodes is substantially reduced, oxidized, or exhausted. 602 occurs prior to the end use application following the first duration of time.

FIG. 7 shows a method 700 of pretreating an electrode, according to a further illustrated embodiment. The method 700 may be used in addition to the method 600 (FIG. 6).

Prior to an end use application and following 602 (FIG. 6), at 702 an electrical potential of the first polarity is applied to the first electrode and an electrical potential of the second polarity is applied to the second electrode. The electrical potentials may apply for a third duration of time, until at least one of the first or second electrodes is substantially oxidized, reduced, or exhausted. While additional pretreatment of electrodes is possible, it currently appears that the effectiveness of pretreatment falls off rapidly after the initial pretreatment.

FIG. 8 shows a method 800 of introducing the first and second electrodes to an electrolytic medium, according to one illustrated embodiment. The method 800 may be employed in the method 500 (FIG. 5).

At 802, the first and second electrodes are placed in a liquid electrolytic medium. For instance, the first and second electrodes may be placed in a bath of electrolytic medium. Such may employ a passive coating mechanism or may employ an active coating mechanism such as electroplating. Alternatively, the first and second electrodes may be placed in a spray or vapor of electrolytic medium. Such may employ a passive coating mechanism or may employ an active coating mechanism such as electrostatic deposition.

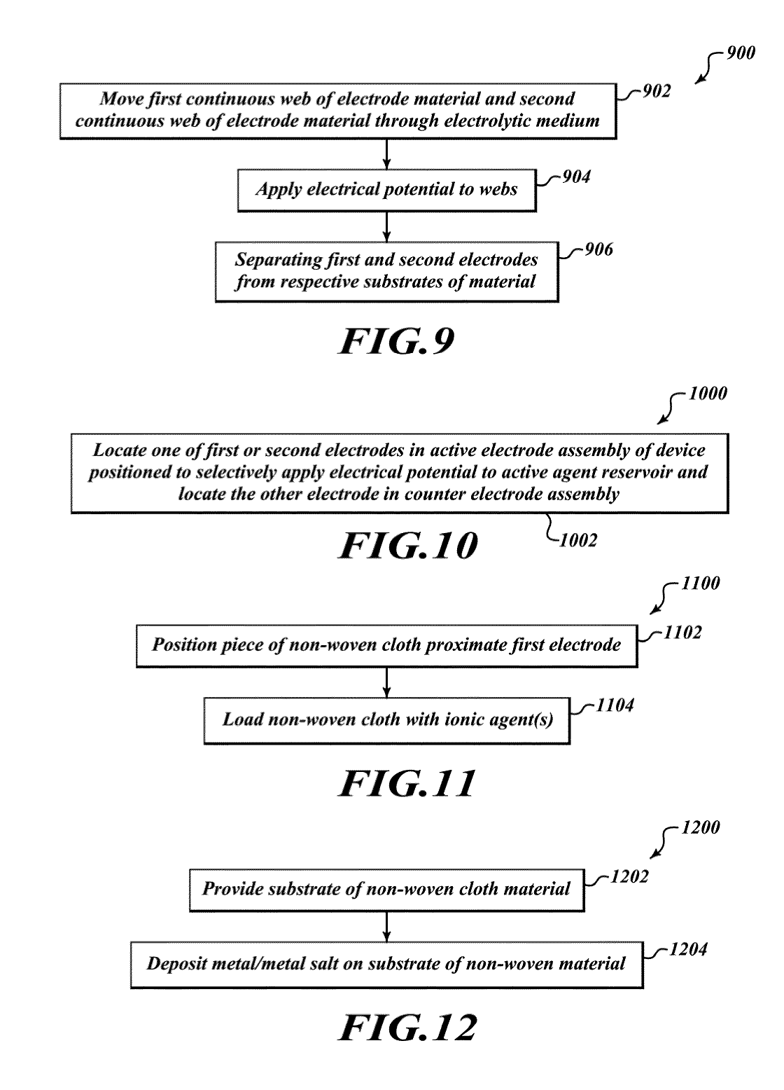

FIG. 9 shows a method 900 of producing electrodes, according to one illustrated embodiment.

At 902, first and second continuous webs of electrode are moved through an electrolytic medium. The continuous webs may be supplied from supply rolls and taken up by take-up rolls, which may be advanced by motors under control of a controller.

At 904, electrical potentials of opposite polarities are applied to the first and second continuous webs of electrode material while in the electrolytic medium. The electrical potentials are applied until at least a portion of at least one of the continuous webs is substantially oxidized, reduced or otherwise exhausted.

At 904, discrete electrodes are separated or otherwise removed from the first and second continuous webs of electrode material. For example, a die-cutter or other type of cutter may cut individual pretreated electrodes from the continuous webs.

FIG. 10 shows a method 1000 of forming a medical device using electrodes, according to one illustrated embodiment.

At 1002, the first or second electrodes are located in an active electrode assembly of an end use device, positioned to selectively apply electrical potential to an active agent reservoir. The other electrode is located in a counter electrode assembly of the end use device. The electrodes may be automatically placed in the end use device, for example via a continuous web manufacturing machinery or via pick and place machinery, etc. Alternatively or additionally, the electrodes may be manually located in the end use device.

While pretreatment is typically discussed herein as occurring before incorporation of the electrodes into an end use device, in some embodiments the electrodes may be pretreated after being incorporated into the end use device. For example, pretreatment of the electrodes may occur after the electrodes are physically associated with a control circuit, but before being physically associated with active agent or drug reservoirs. For instance, such may occur in an end use device in which the active agents or drug reservoirs are selectively insertable into respective receptacles, prior to end use. Also for example, pretreatment of the electrodes may occur after the electrodes are physically associated with an active agent or drug reservoir, but before the active agent or drug is loaded into the reservoir. For instance, such may occur in an end use device in which the active agents or drug reservoirs are selectively loaded into the reservoir, just prior to end use. Thus the claims should not be limited to pretreatment prior to inclusion in the end use device, unless explicitly recited therein.

FIG. 11 shows a method 1100 of forming a portion of a medical device such as an iontophoresis device, according to one illustrated embodiment.

At 1102, a piece of nonwoven fibrous material is positioned proximate a first electrode. Where the nonwoven fibrous material such as an absorbent nonwoven cloth material will hold an ionic agent, the appropriate electrode (i.e., anode, cathode) must be selected based on the polarity of the ionic agent. The nonwoven fibrous material may be automatically placed in the end use device, for example via a continuous web manufacturing machinery or via pick and place machinery, etc. Alternatively or additionally, the nonwoven fibrous material may be manually located in the end use device.

At 1104, the piece of nonwoven fibrous material is loaded with an ionic active agent, for example, oxycodone. The nonwoven fibrous material may be loaded by soaking, spraying or otherwise apply the active agent to the nonwoven fibrous material. The active agent may be automatically loaded in the reservoir, for example via a continuous web manufacturing machinery. Alternatively or additionally, the active agent may be manually located in the reservoir.

FIG. 12 shows a method 1200 of forming an electrode prior to pretreatment, according to one illustrated embodiment.

At 1202, a substrate of nonwoven fibrous material in the form of a nonwoven cloth material is provided. The nonwoven cloth material may be formed of fibers or micro-fibers. The nonwoven cloth material may be absorbent and/or adsorbent. The nonwoven cloth material may take a variety of forms, including natural or non-synthetic materials (e.g., cotton, cellulose) and synthetic material (e.g., rayon).

At 1204, a metal/metal salt is deposited on the substrate of nonwoven cloth material. Numerous examples of suitable metal/metal salt materials have been described, for example Ag/AgCl.

FIG. 13 shows a method 1300 of forming an electrode prior to pretreatment, according to another illustrated embodiment.

At 1302, a metal/metal salt is plated on a substrate of an absorbent nonwoven cloth material. As previously described, the substrate can take a large variety of forms, for example nonwoven fibrous material, foil, screen, or polymer substrate. Conventional plating techniques suitable for the particular substrate material may be employed.

FIG. 14 shows a method 1400 of forming an electrode prior to pretreatment, according to yet another embodiment.

At 1402, a silver/silver chloride ink is deposited on a substrate of absorbent nonwoven cloth material, for example by printing for instance screen printing. Screen printing or other techniques which individually coat fibers making up the nonwoven cloth material may maximize the amount of exposed surface area of the sacrificial material, thereby realizing distinct advantages over other techniques.

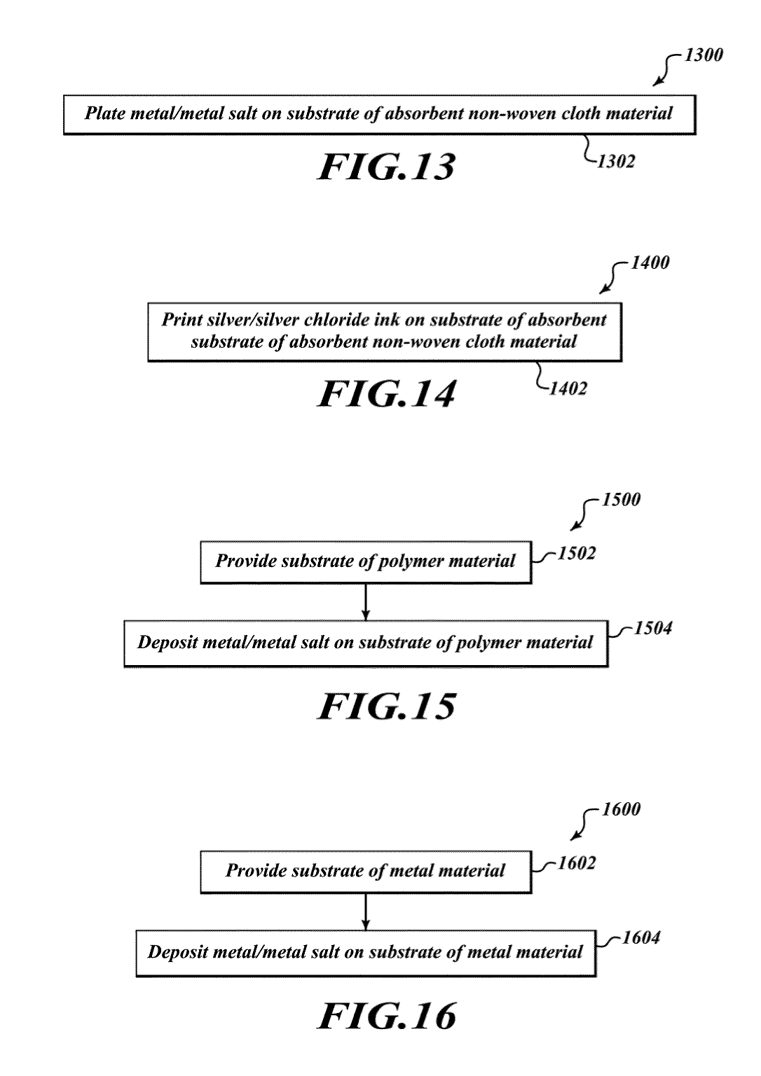

FIG. 15 shows a method 1500 of forming an electrode prior to pretreatment, according to a further illustrated embodiment.

At 1502, a substrate of a polymer material, for instance, PETE is provided. The polymer material may be provided as a continuous web of material to facilitate automated manufacturing using various continuous web production tools and techniques.

At 1504, a metal/metal salt is deposited on the substrate of polymer material. Numerous examples of suitable metal/metal salt materials have been described, for example Ag/AgCl.

FIG. 16 shows a method 1600 of forming an electrode prior to pretreatment, according to still a further illustrated embodiment.

At 1602, a substrate of metal material is provided. As noted previously, the metal substrate material may take the form of foil, screen or plate. The metal substrate material may be provided as a continuous web of material to facilitate automated manufacturing using various continuous web production tools and techniques.

At 1604, a metal/metal salt is deposited on the substrate of metal material. Numerous examples of suitable metal/metal salt materials have been described, for example Ag/AgCl.

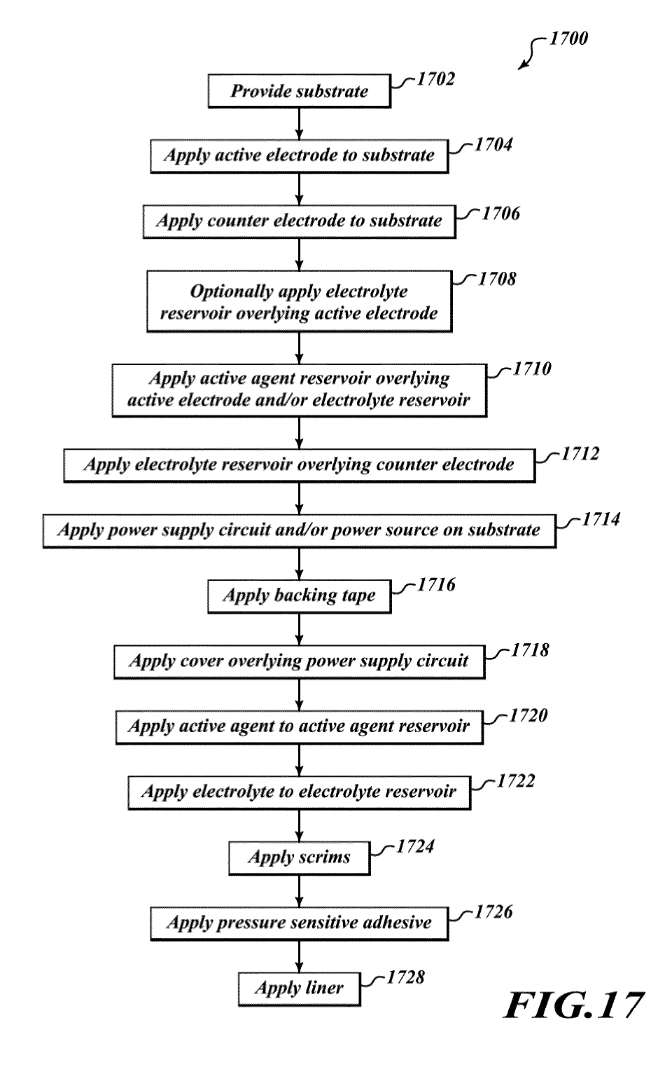

FIG. 17 shows a method of forming a medical device such as a iontophoretic device employing electrodes, according to one illustrated embodiment.

At 1702, a substrate is provided. Numerous examples of suitable substrates have been identified above, including absorbent nonwoven fibrous material.

At 1704, an active electrode is applied to the substrate. Numerous examples of suitable electrochemically active materials have been identified above, including Ag/AgCl.

At 1706, a counter electrode is applied to the substrate. Numerous examples of suitable electrochemically active materials have been identified above, including Ag/AgCl.

In some embodiments, the active and counter electrodes may be applied concurrently or in a different order. The active and counter electrodes are typically applied to a same side of the substrate, which may be denominated as a biological tissue contacting side or “underlying” the substrate.

Optionally, at 1708, an optional electrolyte reservoir may be applied overlying the active electrode. Such may help reduce the occurrence of undesirable reactions or creation of undesirable reactants. The electrolyte reservoir may contain an electrolytic medium, or may later be loaded with an electrolytic medium.

At 1710, an active agent reservoir is applied overlying, aligned with or in registration with the active electrode and/or electrolyte reservoirs. The active agent reservoir is typically an absorbent and/or adsorbent material, which may be synthetic or natural. In some embodiments, the active agent reservoir may take the form of nonwoven fibrous material, which may be the same or different from the nonwoven fibrous that forms the substrate for the electrodes. In other embodiments, the active agent reservoir may take the form of a gel, such as a hydrogel or sol. In still other embodiments, the active agent reservoir may take the form of a container or similar structure.

At 1712, an electrolyte reservoir is applied overlying or aligned with the counter electrode. The electrolyte reservoir is typically an absorbent and/or adsorbent material, which may be synthetic or natural. In some embodiments, the electrolyte reservoir may take the form of nonwoven fibrous material, which may be the same or different from the nonwoven fibrous that forms the substrate for the electrodes. In other embodiments, the electrolyte reservoir may take the form of a gel, such as a hydrogel or sol. In still other embodiments, the electrolyte reservoir may take the form of a container or similar structure.

At 1714, a power supply circuit and/or power source are applied to the substrate. As previously noted, the power supply circuit may take the form of a flexible substrate (e.g., FR4), which may carry one or more components and/or circuit traces on surface thereof or therein. The components may include discrete circuit components (e.g., capacitors, resistors, light emitting diodes, switches) and/or integrated circuit components (e.g., microprocessor, microcontroller, memory or storage, chip based power converters).

At 1716, a secondary substrate, such as a backing tape may be applied. The secondary substrate may have apertures or openings sized and dimensioned to receive respective ones of the reservoirs.

At 1718, a cover may be applied overlying the power supply circuit and/or power source. As previously discussed, the cover may be a polymer, and may provide environmental protection to the various components in the end use device.

At 1720, active agent may be supplied to the active agent reservoir. Various agents that produce a therapeutic or diagnostic effect may be employed as an active agent. For example, oxycodone may be employed. The applicant believes that therapeutically effective administration of oxycodone requires relatively high current densities and relatively long delivery periods (e.g., 24 hours), as compared to other active agents delivered transdermally, for instance LIDOCAINE or LIDOCAINE and epinephrine. Thus, the pretreated electrodes may be particularly suitable for use when the end use application is delivery of oxycodone. Application of the active agent may be automated or manual.

At 1722, electrolyte is applied to the electrolyte reservoir. Various conventional electrolytic mediums may be employed. Application of the electrolyte may be automated or manual.

At 1724, scrims are applied overlying the active agent and electrolyte reservoirs. The scrims may provide protection between the reservoirs and the exterior of the device. Application of the scrims may be automated or manual.

At 1726, a pressure sensitive is applied. Various conventional biocompatible pressure sensitive adhesives may be employed. Application of the pressure sensitive adhesive may be automated or manual.

At 1728, a selectively removable release liner is applied overlying the pressure sensitive adhesive. Various conventional release liners, which are selectively removable from the pressure sensitive adhesive may be employed. Application of the release liner may be automated or manual.

### EXAMPLES

The below description sets out a number of examples, as well as theory about why the superior results may be occurring. Applicant expressly notes that Applicant is not to be bound by such conjectures regarding the theories set out herein, but provides such to allow further research and development.

Applicant believes that pretreatment by running an electrode substantially to exhaustion produces structural changes, which lead to increased electrode performance. While complete exhaustion may produce the best or more significant results, running an electrode to a point short of complete exhaustion may provide meaningful benefit, suitable for some applications and structures.

Applicant believes that increased porosity increases the amount of surface area of the sacrificial material which is exposed to the redox reaction. Applicant believes that such effectively increase the amount of sacrificial material (e.g., Ag/AgCl) while making such material available to the redox reaction. Applicants based such on observations of increased life after pretreatment, scanning electron micrographs of electrodes that were not pretreated and electrodes that were pretreated, measured amounts of Ag/AgCl change, and measured impedance.

Table 1 (below) sets out some observations comparing in electrodes that have no been pretreated to electrodes that have been pretreated by being almost completely exhausted once prior to testing.

TABLE 1
1. Increased life after pre-treatment (300 μA/cm²)

| | No pre-treatment | | | After pre-treatment | |
|---|---|---|---|---|---|
| | (first discharge) | | | (second discharge) | |
| DuPont ink printed onto | 1.47 | hr (n = 4) | [symbol] | 3.2 | hr (n = 2) |
| PETE film Ag/AgCl | | | | | |
| (36.95 mg/cm²) | | | | | |
| DuPont ink printed onto | 7.4 | hr (n = 4) | [symbol] | 15 | hr (n = 2) |
| non-woven cloth Ag/AgCl | | | | | |
| (37.8 mg/cm²) | | | | | |
| DuPont ink printed onto | 11.3 | hr (n = 4) | [symbol] | 27 | hr (n = 4) |
| non-woven cloth Ag/AgCl | | | | | |
| (56.22 mg/cm²) | | | | | |
| Acheson ink printed onto | 1 | hr (n = 4) | [symbol] | 2.3 | hr (n = 2) |
| non-woven cloth Ag/AgCl | | | | | |
| (31 mg/cm²) | | | | | |

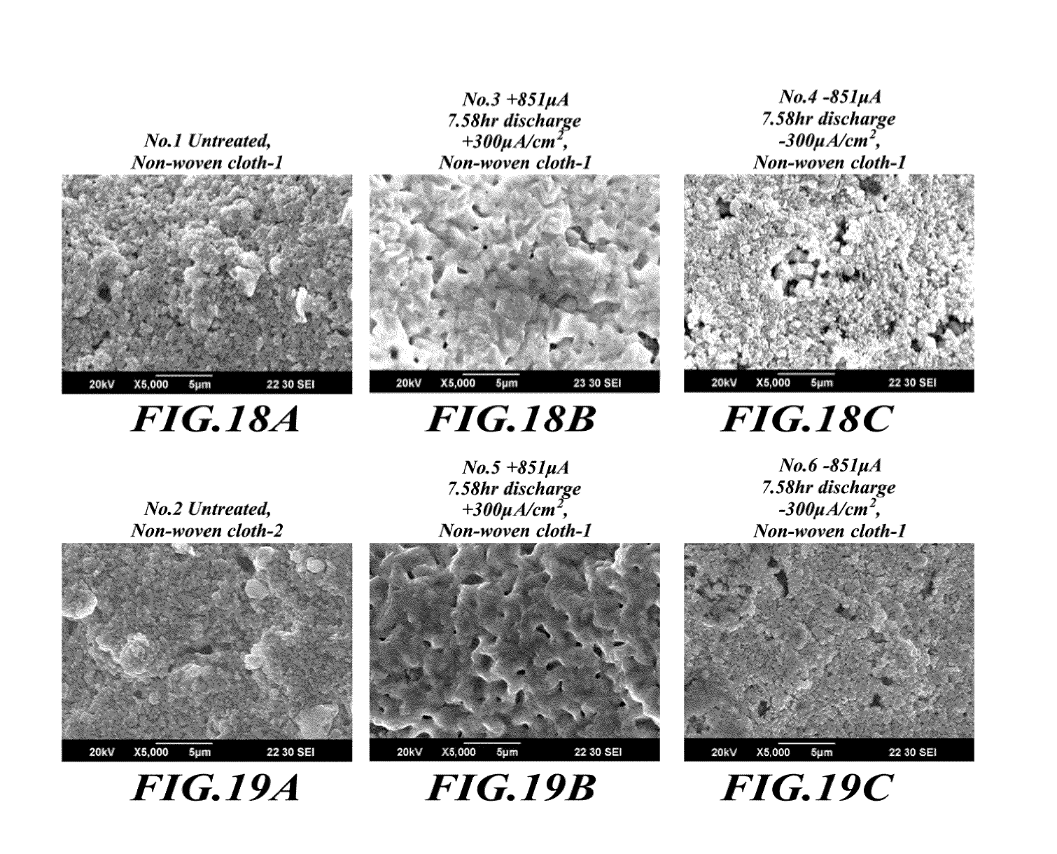

FIGS. 18A-18C are scanning electron micrographs of a first type of untreated nonwoven fibrous or cloth electrode that have not be subjected to pretreatment discharge (i.e., oxidation, reduction or exhaustion).

In particular, FIG. 18A shows the first type of untreated nonwoven fibrous or cloth based electrode prior to any discharge or use. FIG. 18B shows the first type of untreated nonwoven fibrous or cloth based electrode after application of a current of +851 μA at a current density of +300 μA/cm² for 7.58 hours. FIG. 18C shows the first type of untreated nonwoven fibrous or cloth based electrode after application of a current of −851 μA at a current density of −300 μA/cm² for 7.58 hours.

FIGS. 19A-19C are scanning electron micrographs of a second type of untreated nonwoven fibrous or cloth electrode that have not be subjected to a pretreatment discharge.

In particular, FIG. 19A shows the second type of untreated nonwoven fibrous or cloth based electrode prior to discharge or use. FIG. 19B shows the second type of untreated nonwoven fibrous or cloth based electrode after application of a current of +851 μA at a current density of +300 μA/cm² for 7.58 hours. FIG. 19C shows the second type of untreated nonwoven fibrous or cloth based electrode after application of a current of −851 μA at a current density of −300 μA/cm² for 7.58 hours.

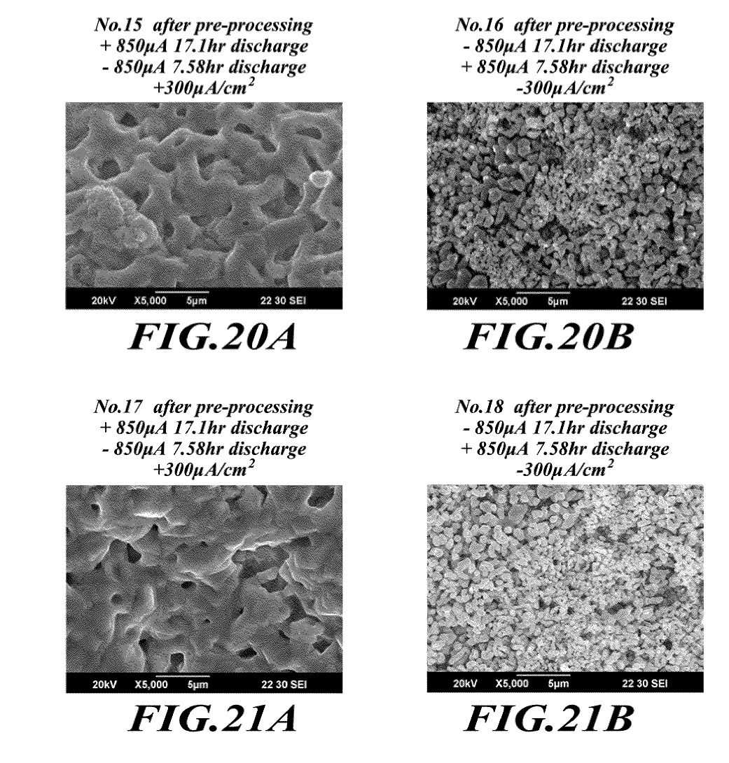

FIGS. 20A and 20B are scanning electron micrographs of a first type of nonwoven fibrous or cloth electrode employing DuPont Ink, that has been subjected to pretreatment.

In particular, FIG. 20A shows the first type of untreated nonwoven fibrous or cloth based electrode that was subject to a pretreatment current of +850 μA at a current density of +300 μA/cm² for 17.1 hours, followed by application of a current of −850 μA at a current density of −300 μA/cm² for 7.58 hours. FIG. 20B shows the first type of untreated nonwoven fibrous or cloth based electrode that was subject to a pretreatment current of −850 μA at a current density of −300 μA/cm² for 17.1 hours, followed by application of a current of +850 μA at a current density of +300 μA/cm² for 7.58 hours.

FIGS. 21A and 21B are scanning electron micrographs of a second type of nonwoven fibrous or cloth electrode, that has been subjected to pretreatment.

In particular, FIG. 21A shows the second type of untreated nonwoven fibrous or cloth based electrode that was subject to a pretreatment current of +850 μA at a current density of +300 μA/cm² for 17.1 hours, followed by application of a current of −850 μA at a current density of −300 μA/cm² for 7.58 hours. FIG. 21B shows the second type of untreated nonwoven fibrous or cloth based electrode that was subject to a pretreatment current of −850 μA at a current density of −300 μA/cm² for 17.1 hours, followed by application of a current of +850 μA at a current density of +300 μA/cm² for 7.58 hours.

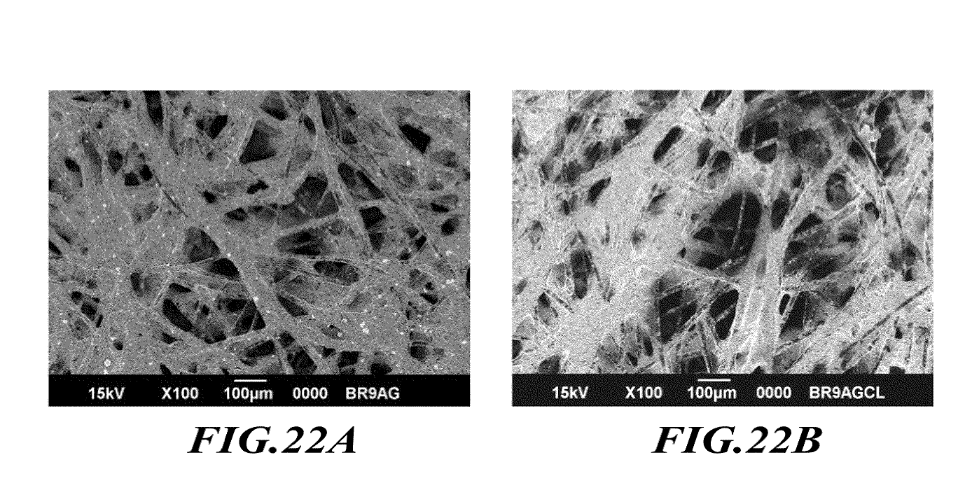

FIGS. 22A and 22B are scanning electron micrographs showing ink coating nonwoven fibers.

In particular, FIGS. 22A and 22B show nonwoven fibers coated with an Ag/AgCl ink sold by DuPont as Ag/AgCl Ink No. L-8144. The DuPont Ag/AgCl ink appears to provide better performance than other Ag/AgCl ink, such as Ag/AgCl ink available from Acheson.

Applicant further believes that use of non-woven fibrous material may provide longer life to electrodes. In particular, Applicant believes that nonwoven fibrous material may promote ion movement. Such is based increased electrode life in comparison to polymer substrate type electrodes, scanning electron micrographs, and measurements of impedance.

Table 2 (below) sets out some observations comparing nonwoven fibrous or cloth type electrode to polymer type electrode.

TABLE 2
2. Performance increase when ink applied to non-woven cloth

| | No pre-processing |
|---|---|
| | (first discharge) |
| DuPont ink printed onto PETE film | 1.5 hr (n = 4) |
| Ag/AgCl (37.0 mg/cm²) | |
| DuPont ink printed onto non-woven cloth | 7.4 hr (n = 4) |
| Ag/AgCl (37.8 mg/cm²) | |

Using the same amount of Ag/AgCl ink on PETE substrate and nonwoven cloth substrate lead to a difference in life. Nonwoven cloth electrodes lasted roughly 5 times as long as PETE substrate electrodes. Both nonwoven and PETE substrate type electrodes showed a marked increase in life when pretreated, for example an approximate doubling of life.

Table 3 (below) sets out some observations comparing silver foil type electrodes that were subjected to a first application of current (i.e., no pretreatment), and a third application of current (i.e., two pretreatment cycles of substantially exhausting the electrode prior to the third application which mimics the end use application of the electrode.) In particular, a silver foil electrode starts as essentially 100% Ag. After a first application of current of a first polarity for a first duration the electrode becomes almost 100% AgCl. After a second application of current of a second polarity, opposite the first polarity, for a second duration the electrode becomes almost 100% Ag. After a third application of current for a third duration, mimicking the end use, the electrode becomes almost 100% AgCl.

TABLE 3
Silver foil life test (300 μA/cm²)

| | | Untreated | | After pre- | |
|---|---|---|---|---|---|
| | | electrode | | treatment | |
| | electrode | (first discharge) | | (third discharge) | |
| Silver foil, 10 um thick | Anode | 8.1 | hr | 5.9 | hr |
| Ag mass 0.031 g | (+) | | | | |
| Silver foil, 20 um thick | Anode | 14.9 | hr | n/a | |
| Ag mass 0.064 g | (+) | | | | |
| Silver foil, 30 um thick | Anode | 15.4 | hr | 17.2 | hr |
| Ag mass 0.092 g | (+) | | | | |
| Silver foil, 50 um thick | Anode | 15.1 | hr | 24.87 | hr |
| Ag mass 0.150 g | (+) | | | | |

Table 4 (below) summarizes observations on electrode life for silver (Ag) electrodes that have had a single pretreatment cycle.

TABLE 4
Electrode life - summary (300 μA/cm²)

| | | After pre- | |
|---|---|---|---|
| | | treatment | |
| | Electrode | (second discharge) | |
| Silver foil 10 um thick | Cathode | 9.3 | hr |
| Ag mass 0.031 g | (−) | | |
| Silver foil 20 um thick | Cathode | 17.8 | hr |
| Ag mass 0.064 g | (−) | | |
| Silver foil 30 um thick | Cathode | 28.46 | hr |
| Ag mass 0.092 g | (−) | | |
| Silver foil 50 um thick | Cathode | 28.3 | hr |
| Ag mass 0.150 g | (−) | | |

Notably, a benefit was realized by pretreatment whether the DuPont or the Acheson Ag/AgCl inks were employed. Comparison was made to electrodes comprising a polyester nonwoven cloth of 19 mm diameter that was screen printed with DuPont Ag/AgCl ink. Test were performed with an electrode of 2.2 grams, Ag/AgCl total of 1.87 grams, of which 1.2 grams was Ag and 0.67 grams was AgCl.

Similar test were performed using silver foil. A 10 mm diameter silver wire was used as a connector, current density was 300 μA/cm². For a foil of 10 μm thickness, an 8 hour life was achieved, while a foil of 20 μm thickness a 16 hour life was achieved.

In at least one test, a first and second electrode each started out with approximately 65% Ag and 35% AgCl prior to any pretreatment. After a first pretreatment or application of electrical potential for a first duration or period, the first electrode has about 30% Ag and 70% AgCl, while the second electrode has approximately 100% AgCl. After a first pretreatment or application of electrical potential for a first duration or period, the first electrode has about 30% Ag and 70% AgCl, while the second electrode has approximately 100% AgCl. Subsequent application of electrical potentials appear to simply swap the ratio of 30% Ag 30% AgCl for one electrode and 100% Ag for the other electrode, back and forth.

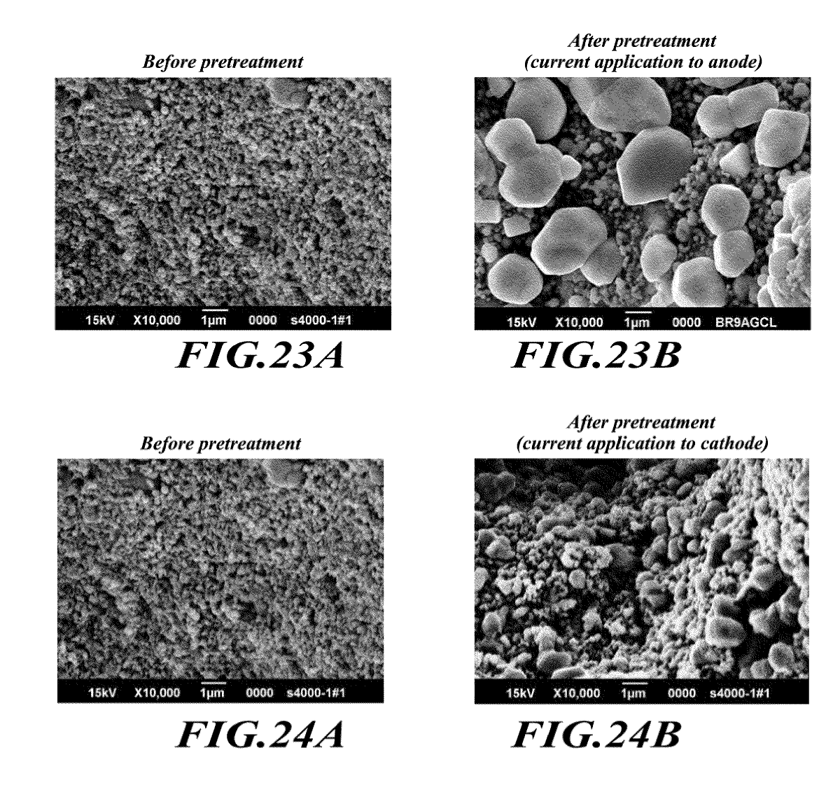

FIGS. 23A and 23B are scanning electron micrographs showing an electrode before and after pretreatment, respectively.

In particular, a deposit of relatively large particles are shown on an anode electrode after application of an electrical potential or pretreatment.

FIGS. 24A and 24B are scanning electron micrographs showing an electrode before and after pretreatment, respectively.

In particular, porosity appears to increase on a cathode electrode after application of an electrical potential or pretreatment.

In at least one set of testing, nonwoven cloth substrates were printed with DuPont Ag/AgCl Ink No. L-8144. The resulting electrodes had a diameter or 19 mm, area of 2.833 cm² and Ag/AgCl mass of 104.7 mg. A constant current of 851 μA at a current density of 300 μA/cm² was applied to the electrodes. The electrodes had lives of approximately 7.51 hours, 7.16 hours. 7.57 hours and 7.36 hours. When subsequently discharged at 300 μA/cm², the electrodes lives had approximately doubled to 15.1 hours, 15 hours. The lives of the remaining two electrodes were not measured, but rather the electrodes were used to produce scanning electron micrographs.

Likewise, PETE substrates were printed with DuPont Ag/AgCl Ink No. L-8144. The resulting electrodes had a diameter or 19 mm, area of 2.833 cm² and Ag/AgCl mass of 107.2 mg. A constant current of 851 μA at a current density of 300 μA/cm² was applied to the electrodes. The electrodes had lives of approximately 1.37 hours, 1.86 hours, 1.33 hours and 1.32 hours. When subsequently discharged at 300 μA/cm², the electrodes lives had approximately 2.1 times increase to 2.91 hours and 3.5 hours 15.1 hours. The lives of the remaining two electrodes were not measured, but rather the electrodes were used to produce scanning electron micrographs.

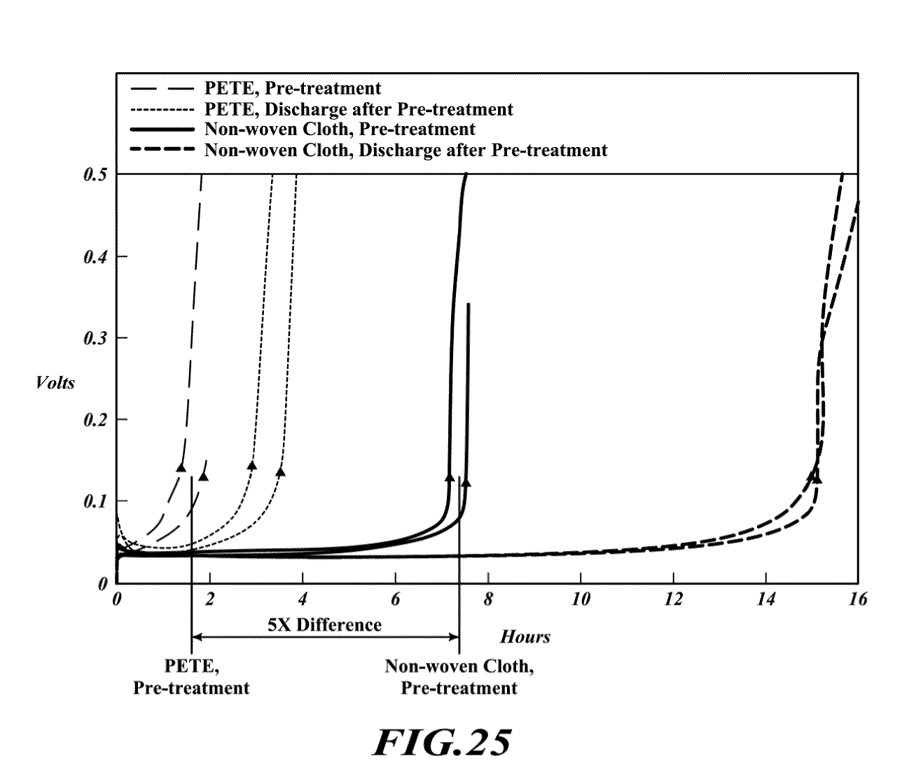

FIG. 25 illustrates the results of the above described testing of nonwoven cloth and PETE substrate type electrodes.

Pretreatment, also referred to as preprocessing or preconditioning, is typically carried out at a constant current density (e.g., 300 μA/cm²). Such may take more time than is desirable for mass production. Hence a number of other processing speeds or current density conditions were investigated. In particular, current densities of 1000 μA/cm², 100 μA/cm², and 300 μA/cm² for pretreatment.

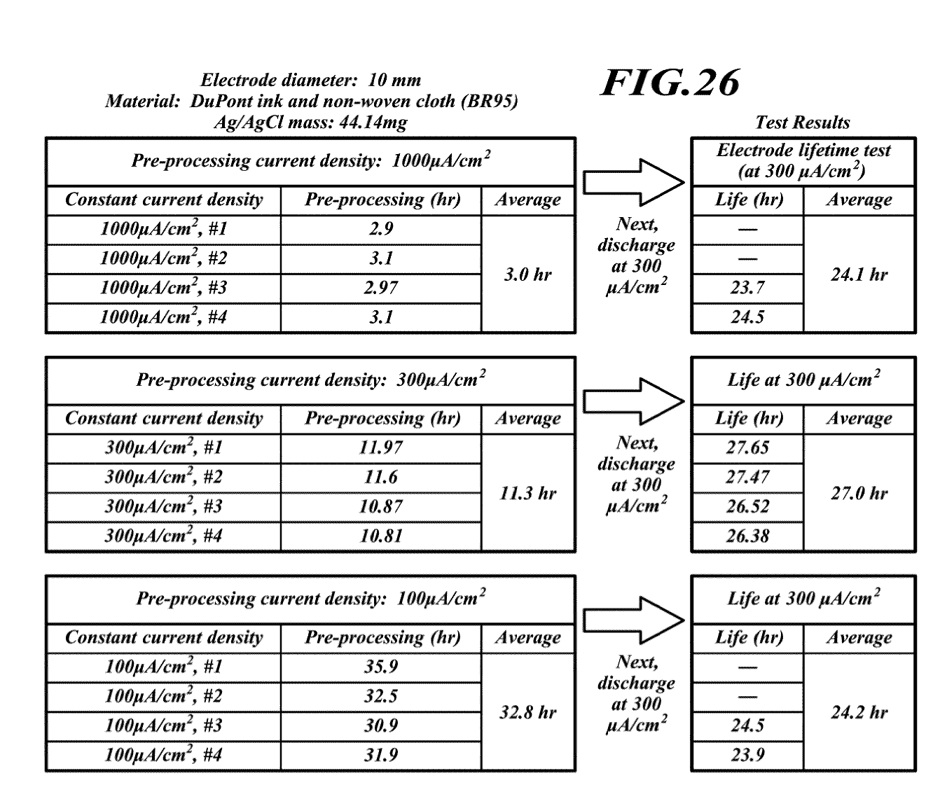

FIG. 26 shows the results of such testing. Although 300 μA/cm² showed better lifetime, the differences between the results for the different current densities appear to be within the margin of error expected with such tests.

The above description of illustrated embodiments, including what is described in the Abstract, is not intended to be exhaustive or to limit the embodiments to the precise forms disclosed. Although specific embodiments of and examples are described herein for illustrative purposes, various equivalent modifications can be made without departing from the spirit and scope of the disclosure, as will be recognized by those skilled in the relevant art. The teachings provided herein of the various embodiments can be applied to other devices that employ electrodes, not necessarily the exemplary medical devices or iontophoretic medical devices generally described above.

The various embodiments described above can be combined to provide further embodiments. To the extent that they are not inconsistent with the specific teachings and definitions herein, all commonly assigned U.S. patents, U.S. patent application publications, U.S. patent applications, foreign patents, foreign patent applications and non-patent publications referred to in this specification and/or listed in the Application Data Sheet, including but not limited to U.S. Patent Application Publication No. 2008-0154178 and U.S. provisional patent application Ser. No. 61/185,544, filed 9 Jun. 2009 are incorporated herein by reference, in their entirety. Aspects of the embodiments can be modified, if necessary, to employ systems, circuits and concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

We claim:

1. A method of manufacture, the method comprising:
prior to an end use application, introducing a first electrode and a second electrode to an electrolytic medium;
prior to the end use application, applying an electrical potential of a first polarity to the first electrode and an electrical potential of a second polarity to the second electrode for a first duration of time until at least one of the first and the second electrodes is substantially oxidized or reduced; and
incorporating the first and the second electrodes into a device for the end use application.

2. The method of claim 1, further comprising:
prior to the end use application and following the first duration of time, applying an electrical potential of the second polarity to the first electrode and an electrical potential of the first polarity to the second electrode for a second duration of time until at least one of the first and the second electrodes is substantially reduced or oxidized.

3. The method of claim 2, further comprising:
prior to the end use application and following the second duration of time and preceding the incorporating of the first and second electrodes into the device for the end use application, applying an electrical potential of the first polarity to the first electrode and an electrical potential of the second polarity to the second electrode for a third duration of time until at least one of the first and the second electrodes is substantially oxidized or reduced.

4. The method of claim 1 wherein introducing a first electrode and a second electrode to an electrolytic medium includes placing the first and the second electrodes in a liquid electrolytic medium.

5. The method of claim 1 wherein introducing a first electrode and a second electrode to an electrolytic medium includes moving a first continuous web of electrode material and a second continuous web of electrode material through the electrolytic medium.

6. The method of claim 1 wherein the end use application is delivery of an active agent to a biological subject and wherein incorporating the first and the second electrodes into a device for the end use application includes locating one of the first or the second electrodes in an active electrode assembly of the device positioned to selectively apply an electrical potential to an active agent reservoir of the active electrode assembly and locating the other one of the first or the second electrodes in a counter electrode assembly of the device.

7. The method of claim 6, further comprising:
positioning a piece of nonwoven cloth proximate the first electrode; and
loading the piece of nonwoven cloth with an ionic active agent to be delivered in response to an electrical potential applied to the ionic active agent via the first electrode.

8. The method of claim 1 wherein the first and the second electrodes are each part of a respective substrate of material, and further comprising:
prior to the end use application and preceding the incorporating of the first and the second electrodes into the device for the end use application, separating the first and the second electrodes from the respective substrates of material.

9. The method of claim 1, further comprising:
prior to the end use application and preceding the introducing of the first and the second electrodes to the electrolytic medium, providing a substrate of nonwoven fibrous material, and
depositing a metal/metal salt on the substrate of nonwoven fibrous material to form the first electrode.

10. The method of claim 1, further comprising:
prior to the end use application and preceding the introducing of the first and the second electrodes to the electrolytic medium, plating a metal/metal salt on a substrate of absorbent nonwoven fibrous material to form the first electrode.

11. The method of claim 1, further comprising:
prior to the end use application and preceding the introducing of the first and the second electrodes to the electrolytic medium, printing a silver/silver chloride ink on a substrate of absorbent nonwoven fibrous material to form the first electrode.

12. The method of claim 1 wherein incorporating the first and the second electrodes into a device for the end use application occurs after introducing the first and the second electrodes to the electrolytic medium and after applying the electrical potential of the first polarity to the first electrode and the electrical potential of the second polarity to the second electrode for the first duration of time.

13. The method of claim 1 wherein applying an electrical potential of a first polarity to the first electrode and an electrical potential of a second polarity to the second electrode for a first duration of time until at least one of the first and the second electrodes is substantially oxidized or reduced includes applying the electrical potential of the first polarity to the first electrode until a capacity for oxidation or reduction of the first electrode is reduced by at least 50 percent relative to an oxidation or reduction capacity the first electrode before the first duration of time.

14. The method of claim 1 wherein applying an electrical potential of a first polarity to the first electrode and an electrical potential of a second polarity to the second electrode for a first duration of time until at least one of the first and the second electrodes is substantially oxidized or reduced occurs before physically associating any one of the first and the second electrodes with an active agent reservoir.

15. The method of claim 1 wherein applying an electrical potential of a first polarity to the first electrode and an electrical potential of a second polarity to the second electrode for a first duration of time until at least one of the first and the second electrodes is substantially oxidized or reduced occurs before loading an active agent in an active agent reservoir.

16. A device for an end use application, comprising:
a first electrode which has had an electrical potential of a first polarity applied for a duration of time until the first electrode has been substantially oxidized or reduced at least one before the end use application;
a second electrode which has had an electrical potential of a second polarity applied for a duration of time until the second electrode has been substantially oxidized or reduced at least once before the end use application; and
a circuit operable to apply a voltage across the first and the second electrodes during the end use application.

17. The device of claim 16 wherein the first electrode has an electrical potential of the second polarity applied for a duration of time until the first electrode has been substantially reconstituted prior to the end use.

18. The device of claim 16 wherein the first electrode has an electrical potential of the second polarity applied for a duration of time until the first electrode has been substantially reduced for a second time prior to the end use.

19. The device of claim 16, further comprising:
an active agent reservoir positioned on a delivery side of the first electrode.

20. The device of claim 19, further comprising:
an ionic active agent loaded in the active agent reservoir and selective transportable from the active agent reservoir in response to an electrical potential applied by the first electrode.

21. The device of claim 16, further comprising:
an absorbent nonwoven cloth active agent reservoir positioned overlaying a biological subject contacting side of the first electrode.

22. The device of claim 21, further comprising:
an absorbent nonwoven cloth electrolyte reservoir positioned overlying a biological subject contacting side of the second electrode; and
a backing structure that supports the first electrode, the second electrode, the circuit, the absorbent nonwoven cloth active agent reservoir and the absorbent nonwoven cloth electrolyte reservoir.

23. The device of claim 21 wherein the first electrode has an area of at least 40 cm² and is capable of delivering 12 mA for 24 hours when driven by the power source.

24. The device of claim 23 wherein the first electrode comprises a nonwoven cloth and a metal/metal salt material.

25. The device of claim 24 wherein the metal/metal salt material comprises an Ag/AgCl mixture of at least 50 mg/cm³.

26. The device of claim 24 wherein the first electrode comprises at least one of a metal foil or screen and has a thickness of at least 30 μm.

27. A device for an end use application of drug delivery, the device comprising:
a first sacrificial electrode;
a second electrode;
an drug reservoir positioned on a delivery side of the first electrode to be responsive to an electrical potential applied by the first sacrificial electrode to delivery a drug from the drug reservoir to a biological interface; and
a circuit operable to apply a voltage from a power source across the first and the second electrodes during the end use application, wherein the first sacrificial electrode has a capacity of delivering 0.3 mA/cm² for a 24 hour duration when driven by the circuit.

28. The device of claim 27 wherein the first sacrificial electrode is sized to delivery 12 mA for 24 hours when driven by the power source.

29. The device of claim 27 wherein the first sacrificial electrode has an area of at least 40 cm².

30. The device of claim 27 wherein the first sacrificial electrode comprises Ag/AgCl.

31. The device of claim 27 wherein the first sacrificial electrode comprises a piece of nonwoven cloth bearing an Ag/AgCl mixture.

32. The device of claim 27 wherein the first sacrificial electrode comprises a piece of polymer substrate bearing an Ag/AgCl mixture.

33. The device of claim 27 wherein the first sacrificial electrode comprises a piece of nonwoven cloth bearing an Ag/AgCl mixture of at least 50 mg/cm³.

34. The device of claim 27 wherein the first sacrificial electrode comprises a piece of foil having a thickness of at least 20 μm and the first sacrificial electrode has had an electrical potential of a first polarity applied for a first duration of time and an electrical potential of a second polarity applied for a second duration of time until the first electrode has been substantially oxidized at least once and reduced at least once before the end use application.

35. The device of claim 27 wherein the first sacrificial electrode comprises a piece of foil having a thickness of at least 30 μm and the first sacrificial electrode has had an electrical potential of a first polarity applied for a first duration of time and an electrical potential of a second polarity applied for a second duration of time until the first electrode has been substantially oxidized at least once and reduced at least once before the end use application.

36. The device of claim 27 wherein the first sacrificial electrode comprises a piece of foil having a thickness of at least 50 μm.

37. The device of claim 27 wherein the Ag/AgCl mixture has had an electrical potential of a first polarity applied for a first duration of time until the first electrode has been substantially oxidized at least one and has had an electrical potential of a second polarity, opposite the first polarity, applied for a second duration of time until reduced at least once before the end use application.

38. The device of claim 27, further comprising:
a therapeutically effective quantity of oxycodone stored in the drug reservoir.
